SEQUENCE LISTING (2 sequences)

SEQ ID NO: 1 - DNA, 46 bp, Artificial Sequence
  linker DNA
catggagatctatcccgggtaacgcgtaagcggccgcaagaattca

SEQ ID NO: 2 - DNA, 46 bp, Artificial Sequence
  linker DNA
catgtgaattcttgcggccgcttacgcgttacccgggatagatctc

(19) United States
(12) Patent Application Publication
(10) Pub. No.: US 2006/0019396 A1
(43) Pub. Date: Jan. 26, 2006

(54) RETROVIRAL VECTORS WITH ENHANCED EFFICIENCY OF TRANSGENE EXPRESSION AND SAFETY

(75) Inventors: Sung Ho HAHM, Woodstock, MD (US); Dug Keun LEE, Gaithersburg, MD (US); Kwan Hee LEE, Gaithersburg, MD (US); Youngsuk YI, Gaithersburg, MD (US)

Correspondence Address: JHK LAW, P.O. BOX 1078, LA CANADA, CA, 91012-1078, US

(73) Assignee: TISSUEGENE, INC., Gaithersburg, MD (US)

(21) Appl. No.: 11/160,066

(22) Filed: Jun. 7, 2005

Related U.S. Application Data
(60) Provisional application No. 60/578,086, filed on Jun. 7, 2004.

(51) Int. Cl. C12N 15/867

(52) U.S. Cl. 435/456

(57) ABSTRACT

The present application discloses a MoMSV/MoMLV hybrid-based retroviral vector, wherein gag and pol genes are completely removed.

### BACKGROUND OF THE INVENTION

1. Field of the Invention

The present invention is related to vectors. The invention also relates to vectors that are used for gene therapy. The invention further relates to constructing such vectors. The present invention is also related to methods of using the vectors for gene therapy.

2. General Background and State of the Art

Retroviral vectors have several advantages to be used as preferred gene transfer vectors in clinical gene therapy trials. These include their high efficiency of transduction into a variety of cell types and ability to integrate into the host cell chromosome allowing for a relatively stable expression of the incorporated genes (Palu, G. et al., Rev Med Virol. 2000 10 185-202; Hawley, R. G., Curr Gene Ther. 2001 1 1-17; Pfeifer, A. and Verma, I. M., Annu Rev Genomics Hum Genet. 2001 2 177-211; Robbins, P. D. et al., Trends Biotechnol. 1998 16 35-40). In the retroviral vectors currently used, the majority of the protein coding sequences for gag, pol and env genes are removed from the viral backbone making them deficient for viral replication. These three major viral proteins are provided in trans in the vector packaging system, either via co-transfecting plasmid constructs expressing genes for these proteins or from packaging cells in which these genes are pre-integrated into the genome (Danos, O. and Mulligan, R. C., Proc. Natl. Acad. Sci. U.S.A. 1988 85 6460-6464; Miller, A. D., Hum. Gene Ther. 1990 1 5-14). The remaining viral backbone contains minimum sequence necessary for encapsidation of the viral RNA (ψ packaging signal sequences), reverse transcription of the viral RNA and integration of proviral DNA (long terminal repeat regions, the transfer RNA-primer binding site, and a region including the 3′ end of the env gene and the polypurine tract) (Palu, G., Parolin et al., C., Rev Med Virol. 2000 10 185-202).

The majority of retroviral vectors are based on Moloney murine leukemia virus (Mo-MLV) and contain a packaging signal extending to the 5′ coding region of the gag gene (ψ⁺) with a replacement of the ATG initiation codon of the gag gene into TAG termination codon. It is generally believed that a sequence element necessary for an efficient nuclear-cytoplasmic transport of RNA molecules is located within the gag open reading frame (King, J. A., et al., FEBS Lett. 1998 434 367-371), and thus inclusion of this sequence in the extended packaging sequence can increase the viral titer (Armentano, D. et al., J. Virol. 1987 61 1647-1650; Bender, M. A. et al., J. Virol. 1987 61 1639-1646). In the wild type murine leukemia virus, unspliced mRNA is transported into the cytoplasm and is packaged into virion as genomic RNA, and it is also used as a template for translation of Gag-Pol fusion and Gag precursor proteins. On the other hand, Env protein is translated from a processed template RNA produced after splicing of the gag and pol coding sequences. Thus, both spliced and unspliced mRNAs are required at an appropriate proportion for a normal replication of the MLV. In the Mo-MLV-based MFG retroviral vector, a splice acceptor site obtained from the 5′ untranslated region of the env gene is introduced downstream of the extended packaging signal (Krall, W. J., et al, Gene Ther. 1996 3 37-48), and transgene proteins are translated from the spliced mRNA templates. These second-generation retroviral vectors can be produced in appropriate packaging cells with a relatively high viral titer.

It is known, however, that the extended packaging signal (ψ⁺) used in these vectors contains a CTG codon upstream of and in frame with the start codon for gag, which is frequently used to produce larger glycosylated Gag protein in the wild type viruses (Edwards, S. A. and Fan, H., J. Virol. 1979 30 551-563). This CTG codon can also be used in the recombinant virus to produce truncated viral protein with a potential immunogenic problem. In order to prevent this problem and to increase viral titer, Miller and co-workers developed MoMSV (Moloney murine sarcoma virus) and MoMLV hybrid vectors (collectively termed as LN series vectors) by replacing the upstream region of the MoMLV vector including sequences starting from the 5′ LTR down to the TAG termination codon introduced to replace the gag gene initiation codon with an equivalent region of the MoMSV (Miller, A. D. and Rosman, G. J., Biotechniques. 1989 7980-982, 984-986, 989-990). The sequence of MoMSV is highly homologous to MoMLV sequence but does not produce the glycosylated Gag protein.

Although these improved vectors are widely used in a variety of applications, all of these vectors contain residual gag and/or pol coding sequences in the ψ⁺ and the splice acceptor sites, respectively. These residual sequences can be used for the generation of replication competent retroviruses (RCR) via recombination with the homologous sequences of the gag and pol genes introduced in the packaging system. It is possible that such RCR pose safety concerns especially during clinical trials. Thus, there is a need in the art to develop vectors that circumvent this potential safety concern.

### SUMMARY OF THE INVENTION

The present invention is directed to a MoMSV/MoMLV hybrid-based retroviral vector, which is devoid of gag, pol or env gene sequence, such that replication competent retrovirus is not generated via recombination with homologous sequence in a viral packaging system.

The invention is also directed to a retroviral vector described above, wherein the vector comprises in order from 5′ to 3′:
(i) a CMV major immediate early gene promoter/enhancer replacing the U3 region of the 5′ LTR;
(ii) a splice donor; and
(iii) a splice acceptor site from EF1-α or CMV-major immediate early gene.

The retroviral vector may further comprise a multicloning site downstream from the splice acceptor site that can facilitate the insertion of a gene of interest.

The invention is also directed to a method of expressing a gene in a fibroblast cell or a chondrocyte cell comprising inserting into the cell the vector described above.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows schematics of retroviral vectors. MFG is a MoMLV-based vector and contains an extended packaging signal (ψ⁺) and SA site from the env gene 5′ untranslated region. It also contains 3′ end of env coding sequence upstream of the 3′ LTR. pDON-A1 is an MFG-based vector, but all the coding sequences from gag, pol and env are removed and the extended region of the packaging signal is replaced with an SA site taken from the human beta-actin gene intron/exon junction. It also replaces the U3 sequence of the 5′ LTR (Mo-MLV) with ˜420 bp of CMV promoter. LN Vector contains 5′ LTR and the packaging sequence obtained from MoMSV and it also contains extended packaging signal extending to gag coding region. pQCXIN vector is a LN-based vector, but is a self-inactivating (SIN) vector as it contains a deletion in the U3 region of the 3′ LTR. Instead, an internal CMV promoter is used for the expression of the transgene. In order to increase viral titer, QCXIN also uses CMV promoter replacing the U3 sequence of the 5′ LTR. A newly developed vector (Se/Sc vector) is also an MSV/MLV-based vector containing a longer CMV promoter (675 bp) placed in front of the R sequence of the 5′ MSV LTR. An SA site taken from an intron/exon junction of either the chimpanzee EF1-α gene or the human CMV major immediate early gene replaces the extended region of the packaging signal.

FIG. 2 shows efficiency of reporter gene expression in GP2-293 cells. Luciferase activity was measured from GP2-293 cells co-transfected with each retroviral vector DNA and VSV-G plasmid DNA, 48-72 hr after transfection. Luciferase activity is shown in an arbitrary unit determined from triplicate wells (mean±S.E.M).

FIG. 3 shows efficiency of reporter gene expression in transiently transduced NIH-3T3 cells. NIH 3T3 cells were transduced with filtered viral supernatant obtained from GP2-293 cells transfected with each retroviral vector and VSV-G. Transduced cells were grown without selection with G-418, and luciferase activity was measured 48 hr after transduction and expressed in an arbitrary unit as a mean±S.E.M determined in triplicate wells.

FIG. 4 shows efficiency of reporter gene expression in stably transduced NIH 3T3 cells. NIH 3T3 cells were transduced with filtered viral supernatant obtained from GP2-293 cells transfected with each retroviral vector and VSV-G. Transduced cells were grown in the presence of G-418 for 6-7 days and counted for cell number after trypsinization. Luciferase activity was measured and expressed in an arbitrary unit after normalizing for the number of cells, as a mean±S.E.M determined in triplicate wells.

FIG. 5 shows efficiency of reporter gene expression in cloned NIH 3T3 after stable transduction. NIH 3T3 cells were transduced with serial dilutions of filtered viral supernatant and grown in the presence of G-418 for 12-14 days, as they were prepared for the purpose of viral titering. Six well-developed and isolated G-418-resistant colonies were picked from wells containing lowest possible viral titer for each vector sample, and grown for additional 5-6 days in 12 well plates until they become confluent. At the time of harvest, cell numbers were counted and luciferase activity is expressed in an arbitrary unit after normalizing with cell number.

### DETAILED DESCRIPTION OF THE PREFERRED EMBODIMENTS

In the present invention, we disclose construction of MoMSV/MoMLV hybrid-based retroviral vectors with an enhanced transcriptional efficiency after integration into the host genome and with significantly improved safety features in terms of the generation of RCR during the packaging process. In the newly developed vectors, the extended packaging sequence of the conventional LN-series vectors containing 5′ portion of the gag coding sequence was replaced with a heterologous splice acceptor sequence obtained from the intron A/exon2 junction of either the chimpanzee EF1-α gene or the human CMV-major immediate early gene. In the LN vectors, transgenes are expressed from spliced mRNAs using the splice donor site located downstream of the 5′ LTR sequence and a cryptic splice acceptor site located in the extended packaging sequence. It has been reported that the efficiency of translation is higher from the spliced mRNA by removing the packaging sequence that can form a complex secondary structure impeding ribosomal progression during the translation process (Mougel, M. et al., Nucleic Acids Res. 1993 21 4677-4684). The increased level of reporter gene expression from NIH 3T3 single clones stably transduced with our newly developed vectors can be attributed to the more efficient generation of spliced mRNA by the introduction of strong heterologous splice acceptor sites.

On the other hand, an increase in splicing efficiency can also cause a reduction in the availability of full-length mRNAs that are required for packaging into new virions, and thus may result in a reduction in viral titer. This may be the reason for a slightly lower level of viral titer recorded from our newly developed vectors compared to pQCFIN vector. In addition, it has been generally believed that the extended region of the packaging signal containing the gag gene coding sequence is required for an increase in the packaging efficiency (Armentano, D. et al., J. Virol. 1987 61 1647-1650; Bender, M. A. et al., J. Virol. 1987 61 1639-1646). However, recent reports suggest that the adverse effect if any of the removal of the extended region of the packaging signal can be overcome by replacing the U3 region of the 5′ LTR with a strong CMV promoter (Yu, S. S. et al., Gene Ther. 2000 7797-804; Naviaux, R. K. et al., J. Virol. 1996 70 5701-5705; Kim, S. H. et al., J. Virol 1998 72 994-1004). In our newly developed vectors, we used the CMV promoter/enhancer sequence extending from −676 bp to −1 from the start of transcription and attached it immediately 5′ to the start of the R sequence of the 5′ LTR. This ensures a higher level of expression of the full-length messages from the vector in the packaging cells, and thus titers from these vectors were maintained at a relatively high level. Furthermore, gag and pol expressing derivative of adenovirus 5-transformed human embryonic kidney 293 cell line (GP2-293) used for packaging recombinant retroviruses in our experiment expresses E1A protein which can mediate high level of transcription from the CMV promoter (Metcalf, J. P. et al., Am. J. Respir. Cell Mol. Biol. 1994 10 448-452; Gorman, C. M. et al., Virology. 1989 171 377-385). This can also help the increase in full-length viral mRNA level and thus the viral titer. Indeed, the efficiency of the reporter gene expression in GP2-293 cells transfected with retroviral vectors was exceptionally high, and luciferase activity was routinely recorded approximately six to seven orders of magnitude higher above the background level. Furthermore, pseudotyping recombinant viruses with VSV-G proteins (Burns, J. C. et al., Proc. Natl. Acad. Sci. U.S.A. 1993 90 8033-8037) enabled us to increase viral titer up to 100 fold above the original level by ultracentrifugation, indicating that the efficiency of transcription rather than the achievement of high titer viral stock is the limiting factor for gene therapy applications.

It is interesting to note that although pQCFIN vector consistently showed higher level of titer than our newly developed vectors, the level of reporter gene expression from cloned NIH 3T3 cells stably transduced with this self-inactivating internal promoter vector was also significantly lower. In line with this observation, while the level of reporter gene expression from pQCFIN transfected GP2-293 cells was approximately 3 times higher than that achieved in cells transfected with Se or Sc vectors, the level of expression in NIH 3T3 cell populations either transiently or stably transduced with pQCFIN was always significantly lower than that achieved with cells transduced with Se or Sc vectors. This indicates that the internal CMV promoter used in pQCXIN vector may not mediate transgene expression as efficiently as the MLV LTR. The increase in the efficiency of transgene expression after incorporation into the host genome as achieved by our newly developed vectors, can thus increase the efficacy of clinical gene therapy trials by lowering the number of virus particles required for the achievement of therapeutically effective level of expression of the protein. This is also important considering the fact that the randomness of incorporation of retroviral vectors into the host genome interfering with the normal expression of the cellular genes at the site of incorporation can become the cause of transforming cells into cancer cells.

As an example, cell-mediated TGF-β1 gene therapy trials via retroviral transduction have been performed successfully for the treatment of artificially induced hyaline cartilage damage in animals (Lee, K. H., et al., Hum. Gene Ther. 2001 12 1805-1813). Application of similar ex-vivo gene therapy protocols in human clinical trials may now require screening for single clones with incorporation of retroviral vectors only at sites of the host genome that do not cause a major interference with the expression of host genes. The increase in transcriptional efficiency from retroviral vectors will allow for the use of the viral supernatant for the transduction of target cells at a lower viral titer, for the purpose of selecting single clones with a stable incorporation with a minimum number of copies of the viral vector. This will not only make the screening procedure easier to perform but also reduce the chance of incorporation of the virus at an undesirable location in the host genome.

Finally, removal of residual gag coding sequences from the extended region of the packaging sequence from our newly developed vectors made them devoid of all the coding sequences for the gag, pol and env genes. This virtually eliminates the chance for the generation of RCR by a recombination process between the homologous regions of the viral vectors and the gag and pol gene sequences introduced in the packaging cells. Our newly developed vectors will thus facilitate helper-free retroviral gene transfer with improved efficacy in clinical gene therapy trials.

Self-Inactivating (SIN) Feature

Another safety concern about retroviral vectors, as for MLV-based vectors, is the possibility of insertional activation of cellular oncogenes by random integration of the vector provirus into the host genome. To overcome this problem, a self-inactivating (SIN) vector may be constructed in which the viral enhancer and promoter sequences are deleted. The long terminal repeat (LTR) in the SIN provirus may be transcriptionally inactivated, which should prevent mobilization by replication-competent virus, for example. This should also enable the regulated expression of genes from internal promoters by eliminating any cis-acting effects of the LTR. Other modifications may be made such as in a vector construct in which the U3 region of the 5′ LTR has been replaced with the cytomegalovirus (CMV) promoter, resulting in Tat-independent transcription. SIN vectors combined with a hybrid 5′ LTR may further reduce the possibility of recombination. These modifications may add additional safety features to the inventive vector system.

The following examples are offered by way of illustration of the present invention, and not by way of limitation.

### EXAMPLES

### Example 1

### Retroviral Vector Construction

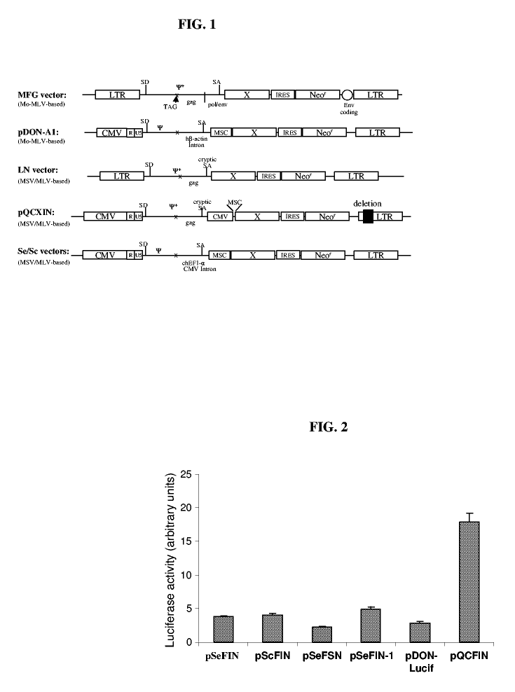

MoMSV-MoMLV hybrid-based retroviral vectors with coding sequences for the gag and pol genes completely removed were made using pLXIN (LN series vector, BD Biosciences) vector as a backbone as shown in FIG. 1. pLXIN vector contains 417 bp of gag coding sequence in the extended region of the packaging sequence (ψ⁺) downstream of the gag gene start codon which was substituted to TAG stop codon. In the newly produced vectors, the gag coding sequence was removed from downstream of the TAG codon and replaced with a sequence flanking a splice acceptor site obtained from the intron A/exon 2 junction of either the chimpanzee EF1-α gene or the human CMV major immediate early gene. In order to study the efficiency of transcription and transduction, firefly luciferase reporter gene was placed downstream of the splice acceptor site. The U5 region of the 5′LTR is completely removed and replaced with an extended CMV major immediate early gene promoter/enhancer (675 bp) in the newly produced vectors, in an attempt to achieve an enhanced expression of the full length viral genome and an increase in viral titer. All these vectors also contain neomycin resistance gene downstream of the reporter gene driven either by the viral LTR through the use of an internal ribosome binding site (IRES) sequence or by a separate SV40 promoter placed downstream of the reporter gene. In order to check the effect of an insertion of a multiple cloning site (MCS) for the convenience of cloning genes in the vector, we also introduced a MCS downstream of the heterologous splice acceptor sites and the luciferase gene was inserted within the MCS site. For the purpose of comparing the efficiency of transduction and transcription, we also used commercially available pDON-A1 (Takara.Mirus.Bio, Madison, Wis.) and pQCXIN (BD Biosciences Clontech, Palo Alto, Calif.) vectors after cloning in the luciferase gene.

### Example 2

### Effciency of the Reporter Gene Expression in GP2-293 Cells Transiently Transfected with Different Retro Viral Vectors

The efficiency of generation of RNA messages to be packaged into viral particles was indirectly estimated by measuring the level of reporter gene expression from the GP2-293 packaging cells co-transfected with retroviral vectors and VSV-G DNA, 48-72 hrs after transfection. The results in FIG. 2 show that comparable levels of reporter gene expression were achieved from the newly produced vectors and the pDON-lucif vector. In contrast to this, the level of expression from the pQCFIN that is a self-inactivating (SIN) vector with an internal CMV promoter was 3-4 times higher than the other vectors. This may be because reporter gene expression from the pQCFIN vector can be driven both by the internal CMV promoter and the CMV promoter placed in the 5′ LTR.

### Example 3

### Effciency of Reporter Gene Expression in NIH 3T3 Cells After Transduction with Viral Supernatant Prepared from GP2-293 Packaging Cells, in the Presence or Absence of G-418 Selection

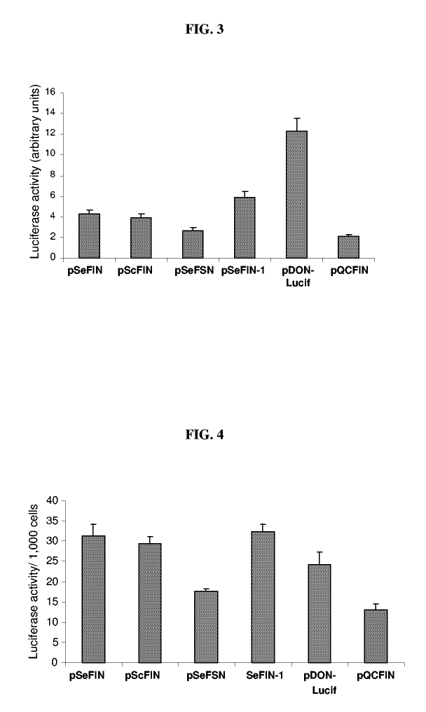

The efficiency of transduction of various retroviral vectors was indirectly estimated by measuring the level of reporter gene expression in NIH 3T3 cells approximately 48 hrs after transduction with viral supernatant prepared from the GP2-293 packaging cells in the absence of selection with G-418. The result in FIG. 3 shows that while the efficiency of transduction of pSeFSN and pQCFIN vectors was 30-40% lower than that of pSeFIN and pScFIN, pDON-lucif showed 4-5 fold higher transduction efficiency than the latter two vectors. The reason for the high level of expression from NIH-3T3 cells transiently transduced with pDON-lucif (without G-418 selection) is not clear because viral titer recorded from this vector preparation was significantly lower than the others (see below). The efficiency of transduction by pSeFIN-1 containing a multiple cloning site downstream of the chimpanzee EF1-α gene splice acceptor site was approximately 50% higher than the same vector without the MCS (pSeFIN), indicating that insertion of the MCS sequence has no detrimental effect on the transduction efficiency.

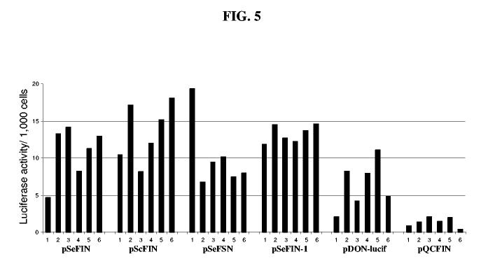

The efficiency of the reporter gene expression from stably transduced NIH 3T3 cells was measured from populations of transduced cells after selecting for transduced cells by G-418. It is shown that while pSeFIN, pScFIN and pSeFIN-1 transduced cells express a comparable level of the reporter gene, pSeFSN, pDON-lucif and pQCFIN transduced cells expressed the reporter gene at 30-50% lower levels (FIG. 4).

### Example 4

### Effciency of Transcription from Stably Transduced Single Clones of Cells

The efficiency of transcription of the reporter gene from various retroviral vectors after stable incorporation into the target cell genome was measured from transduced NIH 3T3 cells after selecting for G-418-resistant single clones. The result in FIG. 5 shows the level of reporter gene expression from 6 clones of transduced cells picked after G-418 selection. For the purpose of studying the efficiency of transcription from the incorporated retroviral vectors, minimizing the effects caused by the incorporation of multi copies of the vector, we picked isolated G418-resistant single colonies of cells formed after transduction with the lowest possible viral titer from the 6-well plates used for titering the virus. As the viral titers in these wells at the time of transduction were at the level lower than 0.001 MOI, we assumed that most of the cells were transduced with a single copy of the vector. Thus, the level of reporter gene expression from these clonal cells likely represents the efficiency of transcription from the single copy incorporated vector. It is shown in FIG. 5 that the efficiency of transcription was variable among clones, reflecting the positional effects anticipated due to the randomness of incorporation of retroviral vectors within the host genome. However, the efficiency of transcription from our Se or Sc vectors (pSeFIN and pScFIN) was consistently higher than the efficiency recorded from pDON-lucif or pQCFIN vector. Also the level of expression from pSeFIN-1 was more uniform among clones and higher than the level achieved from the other vectors. The median expression level from pSeFIN-1 transduced cells (1,325,894 light unit) was approximately 10 fold higher that that achieved from pQCFIN transduced cells (134,593) and more than 2 fold higher than that achieved from pDON-lucif transduced cells (638,103). These results indicate that our newly developed vectors can achieve significantly higher level of expression of the transgene when stably incorporated into the host genome compared with other commercially available retroviral vectors.

### Example 5

### Titering Virus with G-418

The virus titer was determined by growing virus transduced NIH 3T3 cells in the presence of increasing concentrations of G-418 between 0.3 mg/ml and 1 mg/ml. Colonies formed after 12-14 days of G-418 selection were counted under the microscope. This method relies also on the efficiency of the expression of the neomycin resistance gene driven by either an internal SV40 promoter placed downstream of the reporter gene or by the viral promoter (or the internal CMV promoter in the case of pQCFIN) through the use of IRES. Based on this method, our newly developed vectors achieved variable levels of viral titer (Table 1) between 1×10⁴ at the lowest level and up to 1×10⁷ at the highest level. The level of titer achieved by pQCFIN vector (between 2×10⁶ and 1×10⁷) was generally higher than that achieved by the other vectors. On the other hand, pDON-lucif showed more than 3 orders of magnitude lower level of titer compared to the other vectors. The exact reason for this low level of titer from pDON-lucif vector is not clear at this time and it is not consistent with the fact that a highest level of reporter gene expression was observed from NIH 3T3 cells transiently transduced with this vector.

TABLE 1
Determination of viral titer.
Viral titers were determined by counting G-418-resistant NIH 3T3
cell colonies 12-14 days after transduction with retroviral
supernatant in serial dilutions. Ranges of recorded titers
(low and high) are shown determined in 2-5 different experiments
done in duplicates (recorded for the higher titer of the two).

| | Viral Titer | Viral Titer | Viral Titer |
|---|---|---|---|
| | Retroviral Constructs | | |
| | | | After |
| | Low | High | Ultracentrifugation |
| pSeFIN | 1 × 10⁵ | 1 × 10⁷ | 1 × 10⁸ |
| pScFIN | 1 × 10⁴ | 5 × 10⁴ | n.d |
| pSeFS | 2 × 10⁴ | 9 × 10⁶ | n.d |
| pSeFIN-1 | 1 × 10⁴ | 3 × 10⁶ | 5 × 10⁸ |
| pDON-lucif | 2 × 10¹ | 1 × 10² | n.d |
| pQCFIN | 1 × 10⁶ | 1.3 × 10⁷ | 1 × 10⁹ |

As these vectors were pseudotyped with VSV-G proteins, we tried to concentrate and increase the titer of the virus by ultracentrifugation. As shown in Table 1, one time ultracentrifugation of the viral supernatant could increase viral titer 50-100 fold over the original titer, recording a viral titer up to 3×10⁸ for pSeFIN-1.

### Example 6

### Replication Competent Retrovirus Detection

Detection of replication competent retroviruses was performed using an extended S⁺/L⁻ focus-forming assay reported by Cornetta and co-workers (Chen, J. et al., Virology 2001 282 186-197). Replication competent viruses in the supernatant obtained from the packaging GP2-293 cells transfected with viral vectors were first amplified in Mus dunni cells for approximately 3 weeks. M. dunni tail fibroblast cell line was chosen for viral amplification because it is free of endogenous murine leukemia virus sequences and thus unlikely to produce RCR spontaneously (Rigg, R. J. et al., Virology 1996 218 290-295; Lander, M. R. and Chattopadhyay, S. K., J. Virol. 1984 52 695-698). After extended amplification in M. dunni cells, the presence of RCR was determined in S⁺/L⁻ focus-forming assays using PG-4 feline brain cells. According to this method, no RCR was detected in any of the retroviral vector preparations used in this report.

### Example 7

### Vector Construction

Retroviral vectors were constructed using MoMSV-MoMLV-based LXSN vector as a backbone, and the firefly luciferase gene was used as a reporter gene to study the efficiency of transfection and transduction of different constructs. For the insertion of a splice acceptor site either from the chimpanzee EF1-α gene intron A/exon 2 junction or the human CMV major immediate early gene intron A/exon 2 junction, pDRIVE-chEF1 or pDRIVE-hCMV plasmid (InvivoGen, San Diego, Calif.) was used, respectively. The β-galactosidase gene of the pDRIVE plasmids was first removed by cutting with Nco I plus Xba I and replaced with the luciferase reporter gene obtained from pGL-3 plasmid (Promega Corp., Madison, Wis.) Nco I and EcoR I sites, using an EcoR I/Xba I linker (EzcloneSystems, New Orleans, La.). The intron/exon junction sequences were excised from the pDRIVE-luciferase plasmids together with the luciferase gene by digesting with Xho I and EcoR I for the chimpanzee EF1-α sequence and Hpa I and EcoR I for the hCMV sequence and subcloned in the corresponding sites of pCXIN, to produce pSeFIN and pScFIN, respectively. pCXIN was produced by replacing Xho I-Xba I portion of the pCXSN-1 vector with the Xho I-Xba I portion of pLXIN vector (BD Biosciences, San Hose, Calif.). pCXSN-1 was produced from pCXSN by removing the extended packaging signal containing the 5′ coding sequence of the gag gene (Tsp509 I-EcoR I fragment was removed and religated). pCXSN contains extended hCMV enhancer/promoter sequence replaced with the U3 sequence of the 5′ LTR of the pLXSN vector (BD Biosciences, San Hose, Calif.). The 5′ portion of the extended CMV sequence was obtained from pQCXIN (BD Biosciences, San Hose, Calif.) by digesting with Ssp I plus Nde I and subcloned in same sites of the pDON-A1 vector (Takara Bio, Inc., Shiga, Japan) to produce pDON-CMV+. The Bbs I-Bbs I portion of the pDON-CMV+ containing the extended CMV enhancer/promoter sequence was subcloned into the same sites of the pLXSN vector, replacing the U3 sequence. pSeFSN and pScFSN vectors were made by replacing EcoR I-Nhe I portion of the pSeFIN and the pScFIN with the BamH I-Nhe I portion of the pCXSN vector, respectively, using an EcoR I/BamH I linker. For the construction of the pSeFIN-1, a multiple cloning site linker was inserted in the Nco I site located at the start codon of the luciferase gene of the pDRIVE-luciferase construct (pDEFL-1). Sequences for the coding and non-coding strands of the linker DNA were CATGGAGATCTATCCCGGGTAACGCGTAAGCGGCCGCAAGAATTCA (SEQ ID NO:1) and CATGTGAATTCTTGCGGCCGCTTACGCGTTACCCGGGATAGATCTC (SEQ ID NO:2), respectively, and designed to produce inactive Nco I site at the 3′ end after ligation into the Nco I site of the vector. In order to prevent the use of ATG located at the upstream Nco I site of the linker for a false start of translation, the ATG sequence was removed from pDEFL-1 by cutting the vector with Nco I, digesting the protruding ends with mung bean nuclease (Promega), and religating (pDEFL-2). In order to make sure that the luciferase gene contains Kozak sequence 5′ to the start codon, the luciferase gene (Not I-Xba I) of the pDEFL-2 was replaced with the same gene (Hind III-Xba I) from pGL-3, using a Hind III/Not I linker (pDEFL-3). pSEFIN1 was then made by cloning in the Xho I-EcoR I portion of the pDEFL-3 into the same sites of pCXIN-1. pDON-lucif and pQCFIN were made by cloning in the luciferase gene from pGL-3 plasmid (Bgl II-Xba I) into the BamH I-Hpa I sites of pDON-A1 and BamH I-EcoR I sites of pQCXIN after filling in appropriate ends using Klenow DNA polymerase (Promega).

### Example 8

### Production of Retroviral Vector Particles

VSV-G pseudo-typed vector particles were produced by transiently co-transfecting GP2-293 cells with a retroviral vector DNA and VSV-G plasmid. GP2-293 cells were maintained in Dulbecco's modified Eagle's medium (DMEM) supplemented with 10% fetal bovine serum. Cells were transfected in triplicates 16-20 hrs after seeding on collagen-coated 6 well plates at 4-7×10⁵ cells per well, using 6 μl of Fugene-6 (Indianapolis, Ind.) and 1 μg of each DNA per well in a total volume of 1 ml.

### Example 9

### Transduction of NIH-3T3 Cells

NIH 3T3 cells were grown in DMEM containing 1.5 mg/ml of sodium bicarbonate (ATCC, Manassas, Va.) and 10% calf serum in a 5% CO₂ incubator. Cells were seeded at 0.7×10⁵ cells per well in 6 well plates and medium was changed with 1 ml of fresh medium 16-20 hrs after seeding and 1 hr before transduction. For transduction medium from each well of GP2-293, cells transfected with retroviral vectors was filtered through a 0.45 μm polysulfone filter and added directly to a corresponding well of NIH 3T3 cells and a final concentration of 8 μg/ml of polybrene was added. GP2-293 cells were incubated again in a fresh 1 ml D-10 medium. On the next day, medium from NIH 3T3 cells was changed with fresh medium, and cells were transduced one more time with GP2-293 cell virus supernatant as above. After second transduction, GP2-293 cells were washed one time with PBS and harvested for luciferase assay using 0.5 ml of reporter lysis buffer (RLB, Promega). In order to assay for transient transduction efficiency in the absence of neomycin selection, NIH 3T3 cells were harvested in 0.5 ml RLB 48 hr after the second transduction. For measuring stable transduction efficiency in a population of cells after neomycin selection, transduced NIH 3T3 cells were selected for neomycin by adding G-418 in the medium at concentrations between 0.4 mg/ml and 1 mg/ml, starting approximately 36 hrs after the transduction. Cells were harvested 7-10 days after starting G-418 selection.

### Example 10

### Titering Retroviral Vectors

For titering retroviral vectors, NIH 3T3 cells were grown in 6 well plates as above and transduced with virus containing supernatant obtained from GP2-293 cells 48 hr after transfection with retroviral vectors in serial dilutions. For concentrating viral particles, GP2-293 cells were transfected with retroviral vector after grown in 10 cm dishes in 6 ml of D-10 medium. Viral supernatants obtained from two 10 cm GP2-293 cell dishes were pooled together, filtered and centrifuged at 50,000×g in an SW41 ultracentrifuge rotor at 4° C. for 90 min. Virus pellets were resuspended in 30 μl of N-10 medium by shaking at room temperature for 90 min. Ultracentrifuged viruses were diluted 100 times in the same medium before using in the titering experiment as shown above. Approximately 36 hrs after transduction, cells were replaced with G-418 containing medium at increasing concentrations between 0.3 mg/ml and 1 mg/ml and allowed to grow for 12-14 days until distinct colonies are formed, replacing medium every two days. G-418 resistant colonies were counted under the microscope using a 10× objective.

### Example 11

### Determination of Transcriptional Effciency from Transduced G-418-Resistent Single Clones

For the determination of transcriptional efficiency from NIH 3T3 cells transduced with retroviral vectors, 5-6 single clones of transduced cells were picked using 5 mm sterile cloning disks (Sigma-Aldrich Corp, St. Louis, Mo.) according to the manufacturer's protocol. Single colonies were picked from NIH 3T3 plates used for titering retroviral vectors 12-14 days after the start of G-418 selection, from wells inoculated with retroviral supernatant at the highest possible dilution. Colonies picked using the disk were transferred to 12 well plates and allowed to grow for 4 days, and split into fresh 12 well plates at roughly equal densities estimated based on the amount of growth after 4 days. Cells were allowed to grow another 4 days and harvested for the luciferase assay using 500 μl of RLB as above, after counting cell numbers.

### Example 12

### Detection of Replication Competent Retroviruses from Viral Supernatant

RCR test was performed following an extended S⁺/L⁻ assay reported in Chen et al. (2001), which includes 3-week amplification of virus on the permissive Mus dunni cell line and detection of RCR on feline PG-4 cell line by the formation of transformed foci when RCR is present. Both M. dunni cells and PG-4 cells were maintained in McCoy's 5A modified medium supplemented with 10% fetal bovine serum (M-10). M. dunni cells were seeded in T25 flasks at 2×10⁵ cells per flask in 6 ml of M-10 medium one day before transduction, and changed with 3 ml of fresh medium containing 16 μl/ml of polybrene 1 hr before transduction. 3 ml of filtered viral supernatant collected from 3 wells of GP2-293 cells transfected with each retroviral vector for 48 hrs in 6 well plates was then added to the M. dunni cell flask. Cells were allowed to grow for 3 weeks passaging two times per week. After the final passage, cells were allowed to grow additional 2-3 days to become confluent, replaced with fresh medium, and allowed to grow for 1 more day. The supernatant from each flask was collected, filtered through 0.45 μm syringe filter and used in the focus-forming assay. PG-4 cells were seeded in 6 well plates at 1×10⁵ cells per well one day before the assay, and re-fed with 1 ml of medium containing 16 μg/ml of polybrene just prior to the inoculation. One ml of filtered supernatant from M. dunni cells was then added to PG-4 cells (in duplicate) and the formation of discernible foci was checked under the microscope 4-5 days after the inoculation.

All of the cited references are incorporated by reference herein in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A MoMSV/MoMLV hybrid-based retroviral vector, which is devoid of gag, pol or env gene sequence, such that replication competent retrovirus is not generated via recombination with homologous sequence in a viral packaging system.

2. The retroviral vector of claim 1, wherein the vector comprises in order from 5′ to 3′:
(i) a CMV major immediate early gene promoter/enhancer replacing the U3 region of the 5′ LTR;
(ii) a splice donor; and
(iii) a splice acceptor site from EF1-α or CMV-major immediate early gene.

3. The retroviral vector of claim 1, which further comprises a multicloning site downstream from the splice acceptor site that can facilitate the insertion of a gene of interest.

4. A method of expressing a gene in a fibroblast cell or a chondrocyte cell comprising inserting into the cell the vector according to claim 1.
